(19) United States
(12) Patent Application Publication
(10) Pub. No.: US 2025/0375624 A1
(43) Pub. Date: Dec. 11, 2025

(54) Gantry for Therapy Using Fast Neurons and Associated Systems and Methods

(71) Applicant: Fermi Research Alliance, LLC, Batavia, IL (US)

(72) Inventor: Thomas K. Kroc, Batavia, IL (US)

(21) Appl. No.: 18/737,049

(22) Filed: Jun. 7, 2024

(51) Int. Cl. A61N 5/10

(52) U.S. Cl. CPC A61N 5/1081; A61N 5/1042; A61N 2005/109

(57) ABSTRACT

Beam delivery and/or beam aiming subsystems included in a fast neutron therapy system. The beam delivery subsystem comprises a beamline having linear and curved portions. Quadrupoles along the beamline focus particles conveyed between an axled input and a neutron source (e.g., beryllium). A bend magnet(s) directs the particles to collide with the neutron source to release neutrons. A gantry (e.g., concrete, steel) includes an annular rim and opposing annular flanges that form a radial cavity and a perimeter “shield zone” channel. A collimator assembly (e.g., steel) projects radially inward from a slot void in the annular rim. A secondary collimator (e.g., steel, hydrogenous material) presents a barrel extending radially toward the gantry isocenter. The two collimators contour the neutrons into a high linear energy transfer (LET) beam. A drive rotates the gantry to set the delivery angle over approximately 360 degrees about the isocenter (e.g., patient table).

### STATEMENT REGARDING FEDERALLY SPONSORED RESEARCH OR DEVELOPMENT

The invention described in this patent application was made with Government support under the Fermi Research Alliance, LLC, Contract Number DE-AC02-07CH11359 awarded by the U.S. Department of Energy. The Government has certain rights in the invention.

### FIELD OF THE INVENTION

The present invention relates generally to external beam radiation therapy technology and, more particularly, to systems and methods of using a rotating gantry to deliver individualized fast neutron therapy to patients.

### BACKGROUND OF THE INVENTION

External radiation beam therapy is a method of cancer treatment that involves automation designed to selectively direct radiation into cancerous tumors within the body of a patient. A common goal of known approaches to this family of local treatment techniques is irradiating a target tumor such that cancerous cells cease replicating and die while minimizing collateral damage to nearby healthy cells. The particles used in various external radiation beam therapies may be classified according to their Linear Energy Transfer (LET), which is a measure of the density of ionizations along a radiation beam. Higher LET radiations (e.g., alpha particles, neutrons, and heavier ions such as carbon) produce more severe damage to a target tumor, but also to its surrounding healthy cells, than lower LET radiations (e.g., electrons, gamma rays, x-rays).

Certain cancerous tumors (e.g., prostate cancer) that are radioresistant to low LET treatment (largely due to the physical nature of the interactions of photons) may be effectively treated using high LET neutron radiation such as classical fast neutron therapy (FNT)). A typical FNT treatment involves first producing neutrons. Neutrons may be produced by accelerating protons with an accelerator system (e.g. cyclotron, synchrotron, or linear accelerator), and then beaming the protons into a neutron source (e.g., beryllium slug, lithium slug) which causes the system to emit neutrons. The travel paths of these emitted neutrons are shaped into a controlled beam that may be aimed at the target tumor. In this FNT therapy process, the distance between the neutron source and the center of the tumor affects how much healthy tissue near the tumor is also negatively impacted by the therapy. Careful tailoring of the neutron field may minimize damage to healthy cells and maximize therapeutic effect on cancerous cells.

As a matter of definition, a collimator is a beam tailoring device that may filter and shape a particle field while also shielding nearby humans (e.g., patient, therapists) from exposure to particles not controlled within the useful irradiating beam. Properly shielding the beam creation process and choosing an appropriate material for shielding are imperative for treatment system success. As FNT systems are highly cost-intensive, using a cost-effective material to implement effective shielding can greatly reduce the overall system cost.

Another important consideration of FNT system design is means of selective targeting of the neutron beam for individual treatment of a tumor in the patient. A common goal for beam delivery solutions in the field is to direct a neutron beam at the center of a tumor from multiple angles. One known solution is a system comprising a stationary particle beam under which a patient is physically moved to establish a desired treatment angle(s). One problem with this solution is that clinicians are slow to use systems that require a patient to stand or sit upright, and instead prefer to have a patient remain still while the beam targeting system moves about the patient much like the operation of common low LET treatment devices.

Lastly, the energy of the neutron beams that are directed at tumors has a direct impact on treatment effectiveness. The energy of the neutrons delivered by a FNT solution can be said to roughly correlate to the depth of penetration of the dosage. Generally, using lower energy neutrons risks only dosing tissue at shallow depths and not close enough to the tumor to effectuate treatment. Using higher energy neutrons may penetrate tissue to the depth of the tumor but can incur less differentiation in treatment between tumor and healthy tissue.

In summary, known FNT designs commonly present challenges, such as the following:
Cost-effectiveness of design, including material considerations for proper shielding about a neutron beam when aimed at a nonstationary target (e.g., patient)
Delivery of the required neutron energy to efficiently treat a specific tumor
Establishing and maintaining an effective distance from the neutron source to a target treatment site

Accordingly, a need exists for a solution to at least one of the aforementioned challenges in FNT system design and, more specifically, for improvements in the state of the practice for economically, safely, and efficiently providing FNT to a stationary patient.

This background information is provided to reveal information believed by the applicant to be of possible relevance to the present invention. No admission is necessarily intended, nor should be construed, that any of the preceding information constitutes prior art against the present invention.

### SUMMARY OF THE INVENTION

With the above in mind, embodiments of the present invention are related to a beam delivery subsystem and/or a beam aiming subsystem that may be incorporated into a fast neutron therapy (FNT) system.

The beam delivery subsystem may comprise a beamline having a linear portion configured in charged particle communication with a curved portion. The linear portion is characterized by an axled input configured to receive a plurality of particles. The curved portion is characterized by a slug converter configured to carry a neutron source (e.g., beryllium, lithium) and operable to convert the proton beam into a neutron beam using a primary collimator to radiate a plurality of neutrons from the neutron source. A plurality of quadrupoles distributed along the beamline may be configured to focus the plurality of particles within the beamline as conveyed between the axled input and the slug converter. One or more bend magnets distributed along the curved portion may be configured to direct the plurality of particles to collide with the neutron source to release the plurality of neutrons.

The beam aiming subsystem may comprise a gantry of prestressed concrete, steel and/or hydrogenous material. The gantry may be characterized by an annular rim in mechanical communication along a shared axis with an opposing pair of annular flanges. So configured, the annular rim and the annular flanges may collectively form a radial cavity and a perimeter ring channel (i.e., a shield zone). A patient table may be fixedly positioned in the radial cavity substantially coaxial with an axis of the gantry (i.e., the shared axis of the annular rim and the opposing pair of annular flanges). An axially-oriented channel entry may be formed as a first void in one of the opposing pair of annular flanges proximate a radially-oriented slot void formed as a second void in the annular rim. The primary collimator may be made of a steel material. The slug converter may be received by the radially-oriented slot void in the annular rim and may be oriented radially towards the axis of the gantry.

The gantry may be configured to host a secondary collimator (e.g., a multi-leaf collimator system) as received by the radially-oriented slot void. The secondary collimator may be made of a steel and/or hydrogenous material and may be characterized by a barrel void extending from the primary collimator radially toward the axis of the gantry. The primary and secondary collimators may be configured to contour the plurality of neutrons into a neutron beam. For example, and without limitation, the neutron beam may be of a high linear energy transfer (LET) type (e.g., having a magnitude within a range of 45 to 90 mega electron-volts (MeV)). The shield zone may be configured to encase the bend magnet(s) proximate the axially-oriented channel entry and to receive the slug converter through the radially-oriented slot void and proximate the secondary collimator. In certain embodiments, a distance from the slug converter to the axis of the gantry may be approximately 190 centimeters (cm).

In another embodiment of the present invention, the fast neutron therapy system may further comprise a drive configured to rotate the gantry to position the neutron beam at a delivery angle with respect to the axis of the gantry. So configured, the beam delivery subsystem may define a conical rotation path along the linear portion from the axled input as the drive rotates the gantry to set the delivery angle over approximately 360 degrees about the axis of the gantry. In certain embodiments, the gantry may further comprise at least one platform formed in the annular rim facing radially inward toward the axis of the gantry and configured for vertical standing support in the gantry while positioned at the delivery angle.

These and other objects, features, and advantages of the present invention will become more readily apparent from the attached drawings and the detailed description of the preferred embodiments, which follow.

### BRIEF DESCRIPTION OF THE DRAWINGS

The preferred embodiments of the invention will hereinafter be described in conjunction with the appended drawings provided to illustrate and not to limit the invention, where like designations denote like elements, and in which:

FIG. 1 is a front perspective view of a fast neutron therapy system characterized by a beam delivery subsystem and a beam aiming subsystem according to an embodiment of the present invention;

FIG. 2 is a top forward perspective view of the fast neutron therapy system of FIG. 1;

FIG. 3 is a top rear perspective view of the fast neutron therapy system of FIG. 1;

FIG. 4A is a side perspective view of the fast neutron therapy system of FIG. 1 positioned at an exemplary 0 degrees beam delivery angle;

FIG. 4B is a side perspective view of the fast neutron therapy system of FIG. 1 positioned at an exemplary 180 degrees beam delivery angle;

FIG. 5A is a cutaway top perspective view of the fast neutron therapy system of FIG. 1 as taken through line A-A of FIG. 3;

FIG. 5B is a transparent cutaway top perspective view of a slug converter of the fast neutron therapy system of FIG. 5A;

FIG. 6 is a transparent side perspective view of a fast neutron therapy system according to an embodiment of the present invention positioned at an exemplary 90 degrees beam delivery angle;

FIG. 7 is a transparent front view of a fast neutron therapy system according to an embodiment of the present invention positioned at an exemplary 0 degrees beam delivery angle;

FIG. 8 is a transparent front view of the fast neutron therapy system of FIG. 6 at the exemplary 90 degrees beam delivery angle;

FIG. 9 is a transparent front view of a fast neutron therapy system according to an embodiment of the present invention positioned at an exemplary 180 degrees beam delivery angle; and

FIG. 10 is a flowchart of a method of operating a fast neutron therapy system according to an embodiment of the present invention.

Like reference numerals refer to like parts throughout the several views of the drawings.

### DETAILED DESCRIPTION OF THE INVENTION

The present invention will now be described more fully hereinafter with reference to the accompanying drawings, in which preferred embodiments of the invention are shown. This invention may, however, be embodied in many different forms and should not be construed as limited to the embodiments set forth herein. Rather, these embodiments are provided so that this disclosure will be thorough and complete, and will fully convey the scope of the invention to those skilled in the art.

Although the following detailed description contains many specifics for the purposes of illustration, anyone of ordinary skill in the art will appreciate that many variations and alterations to the following details are within the scope of the invention. Accordingly, the following embodiments of the invention are set forth without any loss of generality to, and without imposing limitations upon, the claimed invention.

As used herein, the word “exemplary” or “illustrative” means “serving as an example, instance, or illustration.” Any implementation described herein as “exemplary” or “illustrative” is not necessarily to be construed as preferred or advantageous over other implementations. All of the implementations described below are exemplary implementations provided to enable persons skilled in the art to make or use the embodiments of the disclosure and are not intended to limit the scope of the disclosure, which is defined by the claims.

Furthermore, in this detailed description, a person skilled in the art should note that quantitative qualifying terms such as “generally,” “substantially,” “mostly,” and other terms are used, in general, to mean that the referred to object, characteristic, or quality constitutes a majority of the subject of the reference. The meaning of any of these terms is dependent upon the context within which it is used, and the meaning may be expressly modified.

Certain embodiments of the fast neutron therapy design of the present invention are now described in detail. Throughout this disclosure, the present invention may be referred to as a fast neutron therapy system, a fast neutron therapy assembly, a fast neutron therapy gantry system, a fast neutron beam delivery (sub) system, a fast neutron beam aiming (sub) system, a gantry, an assembly, a device, a system, a product, and/or a method for irradiating a tumor. Those skilled in the art will appreciate that this terminology is only illustrative and does not affect the scope of the invention. For instance, the present invention may just as easily relate to means to administering low LET external radiation beam therapy.

In general, various embodiments of the present invention may employ a gantry that advantageously may provide shielding and structural support for a particle-fed beamline and a neutron beam creation and targeting mechanism. The gantry may be animated by a drive system configured to rotate the gantry about an axis defined proximate a target (e.g., patient). This rotation capability advantageously differentiates the present invention from known fast neutron therapy systems which suffer from limited aiming capability. The patient support may be independent of the gantry, allowing the positioning of the patient to place a target tumor at a focal spot (also referred to hereinafter as an isocenter defined at an axis of rotation of the gantry) of the delivered neutron beam.

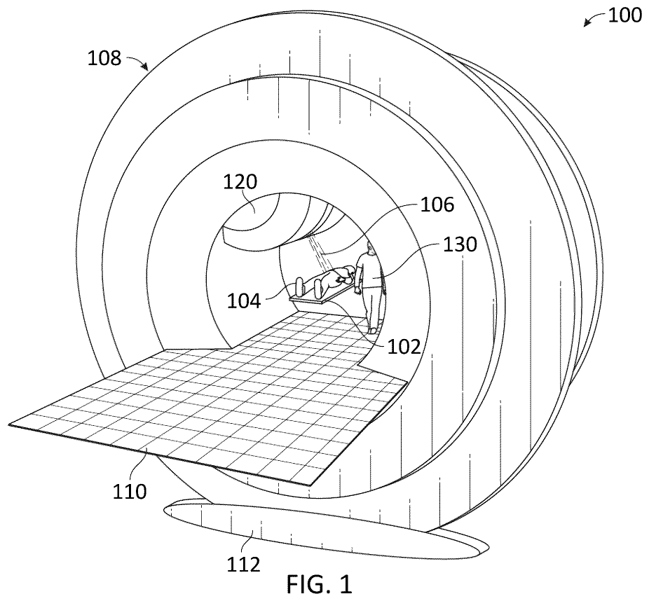

Referring initially to FIG. 1, a fast neutron therapy (FNT) system 100 according to an embodiment of the present invention will now be described in detail. FNT system 100 may comprise a beam aiming subsystem characterized by a substantially “donut-shaped” gantry 108 positioned to axially encircle a fixed target 102 (e.g., patient table). For example, and without limitation, a walking platform 110 proximate the patient table 102 may equip a therapist 130 to stand upright on the walking platform 110 while preparing treatment to a patient 104 lying horizontally on the patient table 102. The gantry 108 may be further configured to carry a collimator assembly 120 operable to direct a neutron beam 106 radially toward the fixed target 102. The gantry 108 may be turned, for example, and without limitation, using a rotator drive 112. As described in more detail hereinbelow, the rotatable gantry 108 so configured may provide both structural support for neutron beam delivery components and shielding from stray radiation generated during the beam creation process.

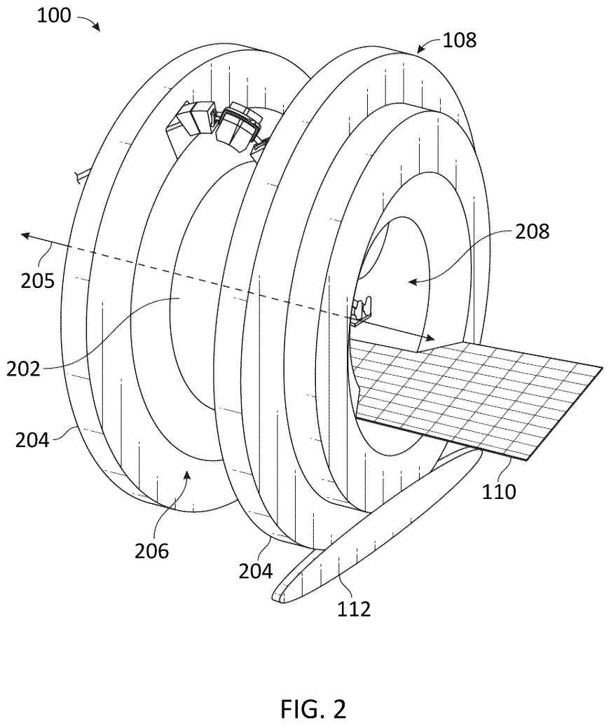

Referring now to FIG. 2, and still referring to FIG. 1, the gantry 108 of FNT system 100 may further comprise an annular rim 202 mechanically connected at respective cylinder ends to one each of a pair of annular flanges 204, The annular rim 202 and “sandwiching” annular flanges 204 may be characterized by a radial cavity 208 that may be large enough to simultaneously accommodate a patient 104, therapist 130, patient table 102, walking platform 110, and/or radially-projecting portion of collimator assembly 120. The annular rim 202 and attached flanges 204 may collectively form a perimeter ring channel referred to hereinafter as a shield zone 206. The rotator drive 112 may be configured to rotate the gantry 108 about an axis 205 shared by the annular rim 202 and paired flanges 204.

The annular rim 202 and/or paired annular flanges 204 may be constructed of a neutron absorber material designed to restrict stray particles (e.g., those particles not part of the desired neutron beam 106) to the shield zone 206. For example, and without limitation, both steel and prestressed concrete may provide adequate shielding from radiation in various embodiments of a gantry 108 of the present invention. As a matter of definition, prestressed concrete is cast around a high-strength steel cable or bar, which is then tensioned. The density of steel may advantageously accomplish the same shielding as concrete in an implemented gantry of the present invention, and with a smaller size compared to prestressed concrete. However, steel may cost significantly more than prestressed concrete material and may require a more complex design of a gantry to accommodate the weight of a steel device. An alternative advantageous embodiment of gantry 108 may therefore employ prestressed concrete to provide required shielding while simplifying the design and/or lowering construction cost. The collimator assembly 120 carried by the gantry 108 may comprise concrete, steel, and/or hydrogenous material.

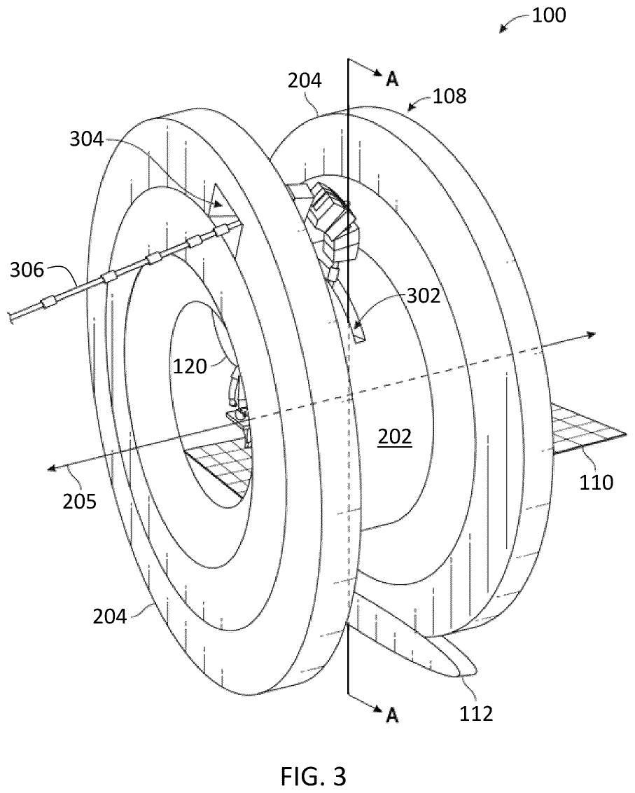

Referring now to FIG. 3, and still referring to FIGS. 1 and 2, FNT system 100 may further comprise a beam delivery subsystem characterized by a beamline 306 that may be configured to project through one of the annular flanges 204 via an axially-oriented channel entry 304 before directing an end of the beamline 306 into the collimator assembly 120 via a radially-oriented slot 302 (also referred to hereinafter as a slot void) in the annular rim 202. In certain embodiments of the present invention, beamline 306 may be configured to receive charged particles (e.g., protons) from a particle source (e.g., a cyclotron, or a linear accelerator) and to direct those particles on a controlled collision course with a neutron source (e.g., a solid mass of a material or “slug,” such as beryllium or lithium, for converting the proton beam into a neutron flux). The distance between the neutron source and the center of a target (e.g., cancerous tumor) is an important consideration because the neutron beam may pass through the tumor and irradiate too much healthy tissue if the distance is too short. A longer distance between the neutron source and the center of the target tumor may reduce the divergence of the beam and may minimize the amount of healthy issue irradiated as the neutron beam passes through the tumor.

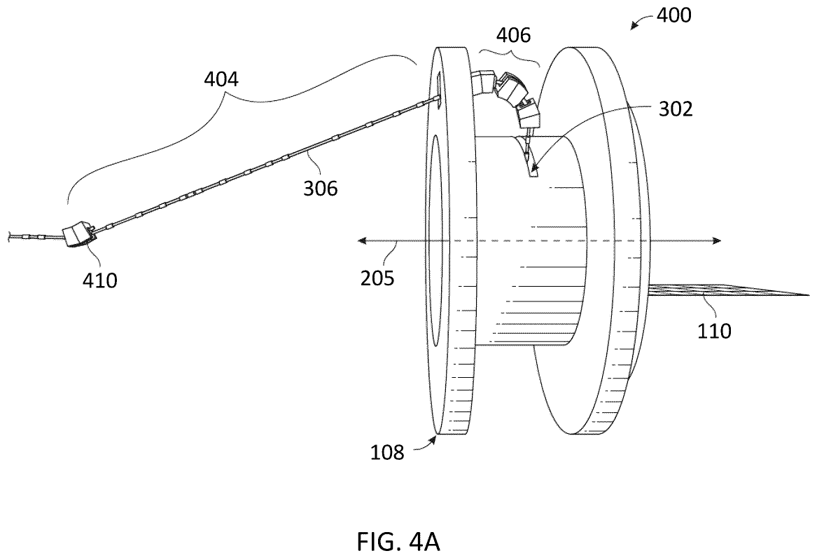

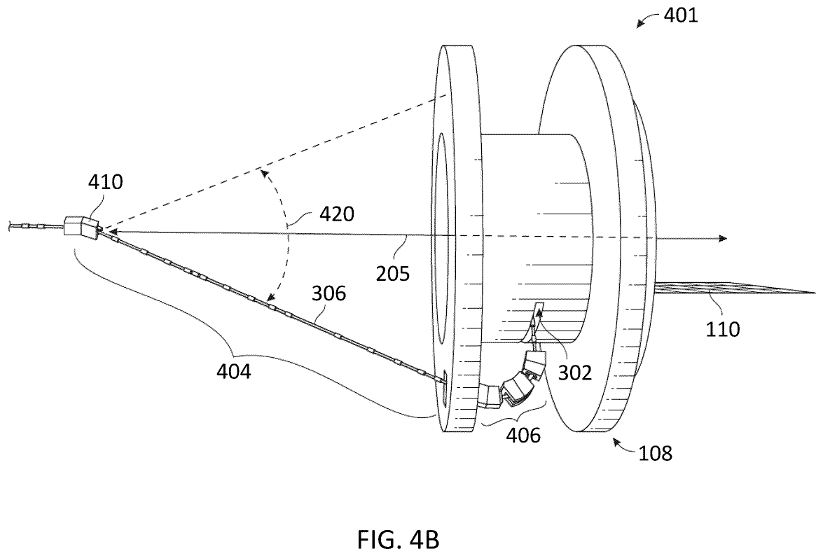

Referring now to both FIG. 4A and FIG. 4B, and still referring to FIGS. 1-3, exemplary beamline 306 may further comprise a linear portion 404 and a curved portion 406. As shown, the linear portion 404 of the beamline may be joined in particle communication with a particle source (not shown) via an axle input 410. Opposite the axle input 410 on the beamline 306, the linear portion 404 may join the curved portion 406 of the beamline 306 proximate the axially-oriented channel entry 304, from which the curved portion 406 of the beamline 306 may continue through the radially-oriented slot void 302 to the collimator assembly 120. So configured, as the gantry 108 of the beam aiming subsystem may rotate, for example, and without limitation, from above the fixed target 102 at the gantry axis 205 (i.e., positioned at 0 degrees as illustrated in schematic 400 of FIG. 4A) to below the fixed target 102 (i.e., positioned at 180 degrees as illustrated in schematic 401 of FIG. 4B), the linear portion 404 of the beamline 306 may define a conical rotation path 420 between the axled input 410 at a first end of the linear portion 404 and the interface point with the curved portion 406 proximate the axially-oriented channel entry 304 of the rotating gantry 108.

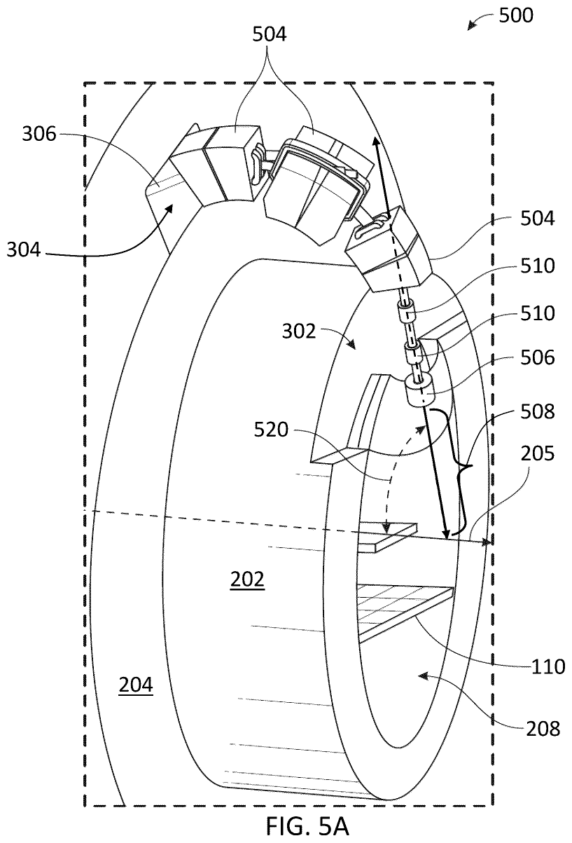

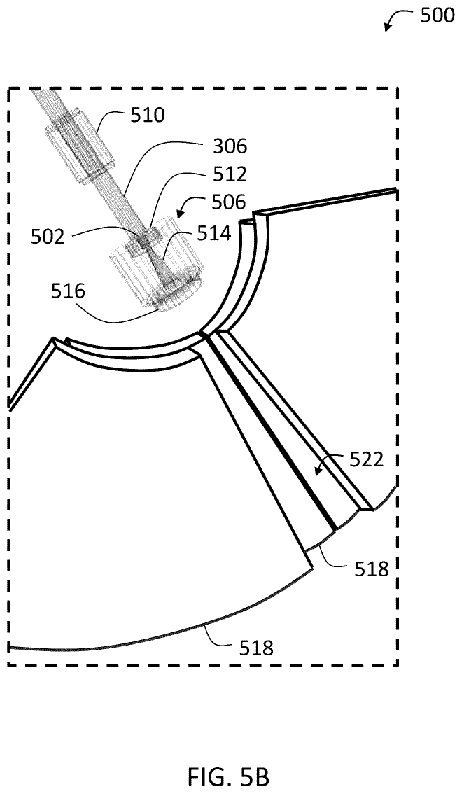

Referring now to FIGS. 5A and 5B, and still referring to FIGS. 1, 2, 3, 4A and 4B, cutaway view 500 of the FNT system 100 illustrates how various components of both the beam delivery subsystem and the beam aiming subsystem described hereinabove cooperate to produce and project neutron beam 106 at a desired delivery vector with respect to the axis 205 of the gantry 108 (e.g., with respect to a fixed target 102). To shape particles traversing the beamline 306, various points along the entire beamline 306 may be adorned with quadrupoles 510 configured to define a collision course with a neutron source 502 carried within a slug converter 506. For example, and without limitation, the substantially cylindrical slug converter 506 may be positioned at a termination of the curved portion 406 of the beamline 306 and may comprise a holder portion 512 configured to provide mechanical support for the neutron source 502 and temperature control (e.g., water cooling channels) during neutron production. In certain embodiments, the slug converter 506 may further comprise an ion chamber 516 configured to measure neutron flux output. The curved portion 406 of the beamline 306 may be adorned with one or more bend magnets 504 that may operate to enforce the particle flow turn toward the neutron source 502 within the slug converter 506 at any point of rotation of the gantry 108. The distance 508 between the slug converter 506 and the axis 205 of the gantry 108 in this example may be interpreted as the distance 508 between the slug converter 506 and the center of a targeted tumor in a patient 104. In certain embodiments of the present invention, the neutron source 502 carried by the slug converter 506 may be a beryllium slug approximately one inch in diameter and approximately one inch long. In such an embodiment, distance 508 may be approximately 190 centimeters (cm).

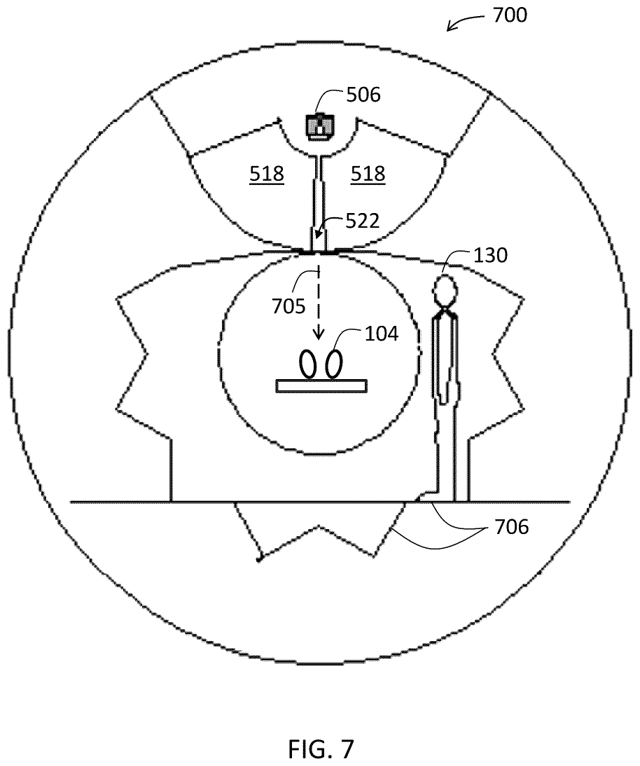

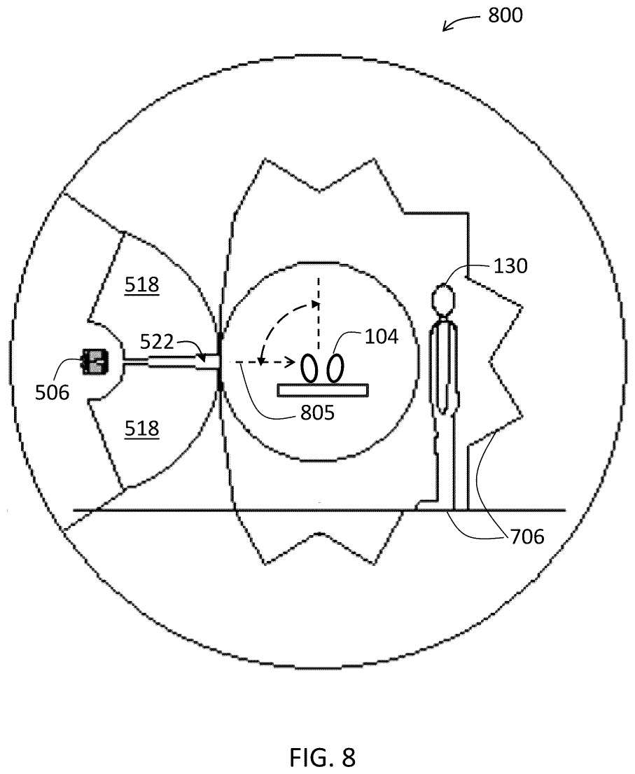

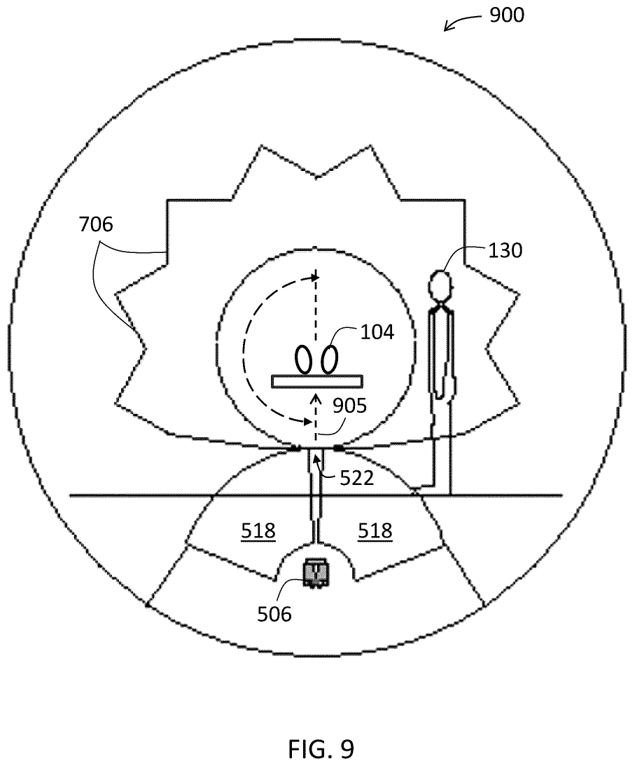

Still referring to FIGS. 5A and 5B, and referring additionally to FIGS. 7, 8 and 9, in certain embodiments of the present invention the slug converter 506 may further comprise a primary collimator 514 that may present, for example, and without limitation, a substantially conical transmission channel extending from the neutron source 502 and axially through the slug converter 506. In certain embodiments, the primary collimator 514 of the present invention may be made from steel. The slot void 302 may be configured to receive and position the slug converter 506 to aim the neutron flux exiting the primary collimator 514 into the collimator assembly 120 for further shaping of the neutron beam 106. The collimator assembly 120 may present one or more beam shaping mechanisms. For example, and without limitation, the collimator assembly 120 may comprise a secondary collimator (e.g., a fixed barrel insert, not shown) configured to be fittedly received by the slot void 302 and positioned to receive and further shape the neutron flux from the primary collimator 514 of the slug converter 506. Also for example, and without limitation, the collimator assembly 120 may comprise a multi-leaf collimator 518. As a matter of definition, a multi-leaf collimator is characterized by individual “leaves” that may move back and forth to create a user-defined contour of a neutron beam (e.g., an adjustable-radius barrel 522) to irradiate a tumor while minimizing radiation exposure to healthy areas of the patient's body. The multi-leaf collimator's leaves also may act as shields in shaping the neutron beam to be projected. The leaves may be made from various shielding materials, such as low carbon steel or polyethylene. In certain embodiments, the multi-leaf collimator 518 of the present invention may be made from hydrogenous material.

As illustrated in FIGS. 3, 4A, 4B, 5A and 6, the slot void 302 may be characterized by an arc length sufficient to receive a fixed barrel insert (not shown) and/or a multi-leaf collimator 518. A person of skill in the art will immediately recognize that less girthy secondary collimator mechanisms may be supported by slot void 302 designs of shorter arc length (e.g., limited only by a radius of the slug converter 506 received by the slot void 302).

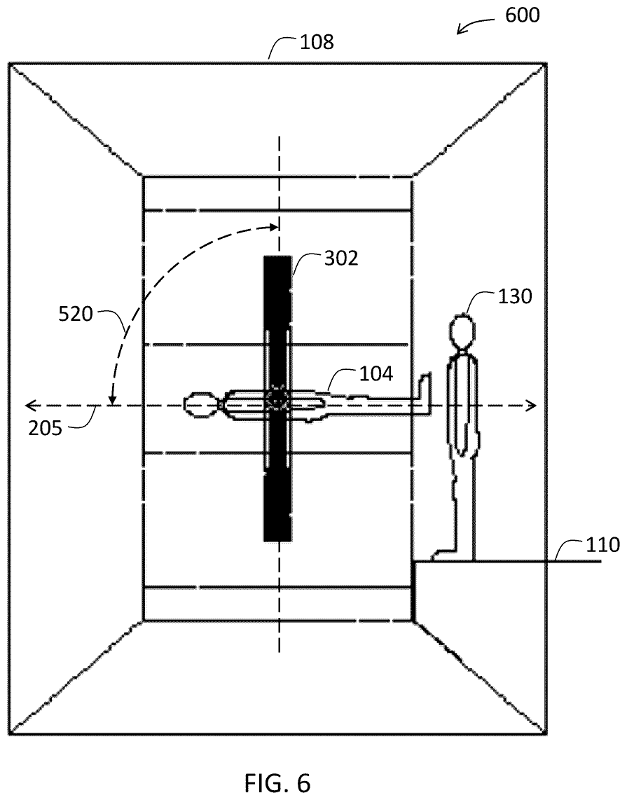

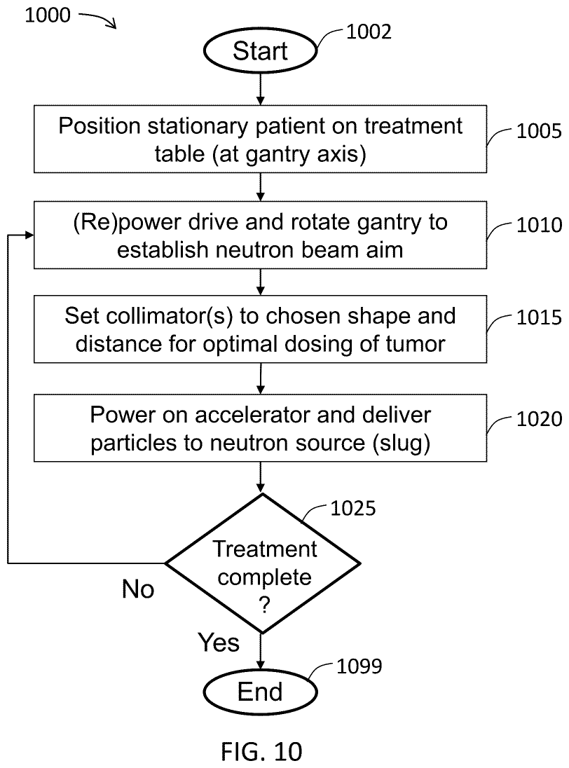

Referring now to FIG. 10, and referring additionally to FIGS. 6-9, a method 1000 of operating the exemplary FNT system 100 of the present invention will now be described in detail. Generally speaking, setting up a patient for treatment may involve aligning the patient to external fiducials (e.g., a visible light beam that mimics the treating radiation beam). Alignment procedures that use either supplementary x-ray or the treating neutron beam itself may require the therapist not be in the treatment room while any such fiducial is active. From the start at Block 1002, a patient 104 may lie horizontally inside the gantry 108 during a beam aiming step (Block 1005) with a therapist 130 standing adjacent on the walking platform 110 as shown in schematic 600 of FIG. 6. At Block 1010, the rotating drive 112 may be powered and operated to rotate the gantry 108 to set the delivery vector of the prospective neutron beam 106 (e.g., along the path toward which the curved portion 406 inside the radially-oriented slot void 302 points the slug converter 506 and shapes the beam 106 using both the primary collimator 514 and the secondary (e.g., multi-leaf) collimator 518; as illustrated in FIGS. 5A and 6, the delivery vector of beam 106 may be characterized by an fixed axial angle 520 (e.g., 90 degrees) with respect to axis 205 throughout any rotation of gantry 108 about that axis 205 that positions the beam 106 radially with respect to that axis 205). For example, and without limitation, schematic 700 demonstrates aiming of the neutron beam 106 set at a 0 degrees central angle in relation to stationary patient 104 (i.e., tail and head of delivery vector of beam 106 aligned directly above 705); schematic 800 demonstrates aiming of the neutron beam 106 set at a 90 degrees central angle in relation to stationary patient 104 (i.e., tail and head of delivery vector of beam 106 aligned to a side horizontally 805); and schematic 900 demonstrates aiming of the neutron beam 106 set at a 180 degrees central angle in relation to stationary patient 104 (i.e., tail and head of delivery vector of beam 106 aligned directly below 905). Having positioned the desired central angle (also referred to herein as the delivery angle) of the delivery vector of the prospective neutron beam 106 projecting from the primary collimator 514 of the slug converter 506, the collimator assembly 120 may be further tailored for optimal dosing at Block 1015 (e.g., secondary multi-leaf collimator 518 may be manipulated to form a custom barrel 522 configured to set desired beam shape and/or distance 508 characteristics). At Block 1020, powering on the particle source (not shown) to introduce particles into the beam delivery subsystem as described hereinabove may produce a neutron beam 106 directed at a target (e.g., patient 104) at the set delivery angle, distance 508, and magnitude. If, at Block 1025, planned fast neutron therapy is determined not to be complete, the subprocesses of rotating the gantry 108 (Block 1010), tailoring the collimator assembly 120 (Block 1015) and applying the neutron beam 106 to a treatment area (Block 1020) may be repeated as many times as medically needed. To facilitate patient care and/or FNT system setup and operation, vertical standing points 706 about the gantry 108 may augment the walking platform 110 to allow a therapist 130 to stand vertically regardless of the central angle of the delivery vector of the neutron beam 106 at which the gantry 108 of the delivery aiming subsystem may be temporarily stopped. Upon completion of therapy (Block 1025), the FNT system operation method 1000 may end at Block 1099.

While known systems may employ a fixed horizontal beam directed at a target (e.g., cancerous tumor inside a patient), the proposed system advantageously may employ a neutron beam delivery means that may be rotated around a patient lying horizontally within a gantry. Medical practitioners are known to prefer treatment delivery solutions that allow patients to lie horizontally instead of upright. The present invention may accommodate this preference while employing a safe, effective, and affordable design.

Proposed system advantages include, but are not limited to, the following:
A rotating gantry providing and directing fast neutron therapy to a cancerous tumor
Prestressed concrete within the gantry for cost efficient shielding and structural support

Some of the illustrative aspects of the present invention may be advantageous in solving the problems herein described and other problems not discussed which are discoverable by a skilled artisan.

While the above description contains much specificity, these should not be construed as limitations on the scope of any embodiment, but as exemplifications of the presented embodiments thereof. Many other ramifications and variations are possible within the teachings of the various embodiments. While the invention has been described with reference to exemplary embodiments, it will be understood by those skilled in the art that various changes may be made, and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best or only mode contemplated for carrying out this invention, but that the invention will include all embodiments falling within the scope of the appended claims. Also, in the drawings and the description, there have been disclosed exemplary embodiments of the invention and, although specific terms may have been employed, they are unless otherwise stated used in a generic and descriptive sense only and not for purposes of limitation, the scope of the invention therefore not being so limited. Moreover, the use of the terms first, second, etc. do not denote any order or importance, but rather the terms first, second, etc. are used to distinguish one element from another. Furthermore, the use of the terms a, an, etc. do not denote a limitation of quantity, but rather denote the presence of at least one of the referenced items.

Thus, the scope of the invention should be determined by the appended claims and their legal equivalents, and not by the examples given.

That which is claimed is:

1. A fast neutron therapy system comprising:
a beam delivery subsystem comprising:
a beamline having a linear portion configured in charged particle communication with a curved portion, wherein the linear portion is characterized by an axled input configured to receive a plurality of particles, and wherein the curved portion is characterized by a slug converter comprising a primary collimator configured to radiate a plurality of neutrons from a neutron source carried by the slug converter,
a plurality of quadrupoles distributed along the beamline and configured to focus the plurality of particles within the beamline as conveyed between the axled input and the slug converter, and
at least one bend magnet distributed along the curved portion and configured to direct the plurality of particles to collide with the neutron source to release the plurality of neutrons; and
a beam aiming subsystem comprising:
a gantry characterized by an annular rim in mechanical communication with an opposing pair of annular flanges collectively forming a radial cavity and forming a perimeter ring channel, to define a shield zone, and including an axially-oriented channel entry in one of the opposing pair of annular flanges proximate a radially-oriented slot void in the annular rim extending radially toward an axis of the annular rim of the gantry, and
a secondary collimator received by the radially-oriented slot void and characterized by a barrel void extending radially toward the axis of the annular rim of the gantry and configured to contour the plurality of neutrons into a neutron beam;
wherein the shield zone is configured to encase the at least one bend magnet proximate the axially-oriented channel entry and to receive the slug converter through the radially-oriented slot void and proximate the secondary collimator.

2. The fast neutron therapy system according to claim 1, further comprising a drive configured to rotate the gantry to position the neutron beam at a delivery angle with respect to the axis; wherein the beam delivery subsystem is configured to define a conical rotation path along the linear portion from the axled input as the drive rotates the gantry to set the delivery angle over approximately 360 degrees about the axis.

3. The fast neutron therapy system according to claim 1, wherein the gantry further comprises a patient table positioned in the radial cavity substantially coaxial with the axis of the annular rim of the gantry.

4. The fast neutron therapy system according to claim 1, wherein the gantry further comprises a platform formed in the annular rim facing radially inward toward the axis and configured for vertical standing support in the gantry at the delivery angle.

5. The fast neutron therapy system according to claim 1, wherein the gantry is of a material type selected from the group consisting of prestressed concrete, steel, and hydrogenous material.

6. The fast neutron therapy system according to claim 1, wherein a distance from the slug converter to the axis of the gantry is approximately 190 centimeters (cm).

7. The fast neutron therapy system according to claim 1, wherein the primary collimator is of steel material type and the secondary collimator is of at least one of steel material type and hydrogenous material type.

8. The fast neutron therapy system according to claim 1, wherein the neutron beam is of a high linear energy transfer (LET) type.

9. The fast neutron therapy system according to claim 8, wherein the neutron beam is of a magnitude range of 45 to 90 mega electron-volts (MeV).

10. A beam delivery subsystem for use with a fast neutron therapy system comprising a beam aiming subsystem comprising:
a gantry characterized by an annular rim in mechanical communication with an opposing pair of annular flanges collectively forming a radial cavity and forming a perimeter ring channel, to define a shield zone, and including an axially-oriented channel entry in one of the opposing pair of annular flanges proximate a radially-oriented slot void in the annular rim,
a collimator assembly extending from the radially-oriented slot void of the gantry radially toward an axis of the annular rim of the gantry, and
a secondary collimator received by the radially-oriented slot void and characterized by a barrel void extending radially toward the axis of the annular rim of the gantry;
the beam delivery subsystem comprising:
a beamline having a linear portion configured in charged particle communication with a curved portion, wherein the linear portion is characterized by an axled input configured to receive a plurality of particles, and wherein the curved portion is characterized by a slug converter comprising a primary collimator configured to radiate a plurality of neutrons from a neutron source carried by the slug converter,
a plurality of quadrupoles distributed along the beamline and configured to focus the plurality of particles within the beamline as conveyed between the axled input and the slug converter, and
at least one bend magnet distributed along the curved portion and configured to direct the plurality of particles to collide with the neutron source to release the plurality of neutrons;
wherein the at least one bend magnet is configured for positioning within the shield zone proximate the axially-oriented channel entry;
wherein the slug converter is configured for positioning through the radially-oriented slot void and proximate the secondary collimator.

11. The beam delivery subsystem according to claim 10, wherein the primary collimator and secondary collimator are cooperatively configured to contour the plurality of neutrons into a neutron beam; and further comprising a drive configured to rotate the gantry to position the neutron beam at a delivery angle with respect to the axis; wherein the beam delivery subsystem is configured to define a conical rotation path along the linear portion from the axled input as the gantry rotates to set the delivery angle over approximately 360 degrees about the axis.

12. The beam delivery subsystem according to claim 11, wherein the neutron beam is of a high linear energy transfer (LET) type.

13. The beam delivery subsystem according to claim 11, wherein the neutron beam is of a magnitude range of 45 to 90 mega electron-volts (MeV).

14. The beam delivery subsystem according to claim 10, wherein the neutron source comprises beryllium.

15. A beam aiming subsystem for use with a fast neutron therapy system comprising a beam delivery subsystem comprising:
a beamline having a linear portion configured in charged particle communication with a curved portion, wherein the linear portion is characterized by an axled input configured to receive a plurality of particles, and wherein the curved portion is characterized by a slug converter comprising a primary collimator configured to radiate a plurality of neutrons from a neutron source carried by the slug converter,
a plurality of quadrupoles distributed along the beamline and configured to focus the plurality of particles within the beamline as conveyed between the axled input and the slug converter, and
at least one bend magnet distributed along the curved portion and configured to direct the plurality of particles to collide with the neutron source to release the plurality of neutrons;
the beam aiming subsystem comprising:
a gantry characterized by an annular rim in mechanical communication with an opposing pair of annular flanges collectively forming a radial cavity and a perimeter ring channel, to define a shield zone, and including an axially-oriented channel entry in one of the opposing pair of annular flanges proximate a radially-oriented slot void in the annular rim,
a collimator assembly extending from the radially-oriented slot void of the gantry radially toward an axis of the annular rim of the gantry, and
a secondary collimator received by the radially-oriented slot void and characterized by a barrel void extending radially toward the axis of the annular rim of the gantry and configured to contour the plurality of neutrons into a neutron beam;
wherein the shield zone is configured to encase the at least one bend magnet proximate the axially-oriented channel entry;
wherein the radially-oriented slot void is configured to receive the slug converter proximate the secondary collimator; and
wherein the gantry is configured to rotate the beam delivery subsystem to define a conical rotation path along the linear portion from the axled input and to set a delivery angle with respect to the axis.

16. The beam aiming subsystem according to claim 15, wherein the gantry further comprises a patient table fixedly positioned in the radial cavity substantially coaxial with the axis of the annular rim of the gantry.

17. The beam aiming subsystem according to claim 15, wherein the gantry further comprises a platform formed in the annular rim facing radially inward toward the axis and configured for vertical standing support in the gantry at the delivery angle.

18. The beam aiming subsystem according to claim 15, wherein the gantry is of a material type selected from the group consisting of prestressed concrete and steel.

19. The beam aiming subsystem according to claim 15, wherein the primary collimator is of steel material type and the secondary collimator is of at least one of steel material type and hydrogenous material type.

20. The beam aiming subsystem according to claim 15, further comprising a drive configured to rotate the gantry to position the neutron beam at the delivery angle.
